(19) United States
(12) Patent Application Publication
(10) Pub. No.: US 2007/0031471 A1
(43) Pub. Date: Feb. 8, 2007

(54) Uses of Amniotic Membranes as Biocompatible Devices

(75) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(73) Assignee: MINU, L.L.C., Pittsboro, NC (US)

(21) Appl. No.: 11/425,017

(22) Filed: Jun. 19, 2006

Related U.S. Application Data
(63) Continuation-in-part of application No. 10/874,724, filed on Jun. 23, 2004.

(51) Int. Cl. A61F 2/00

(52) U.S. Cl. 424/427

(57) ABSTRACT

Amniotic membranes provided as emulsions to provide desirable features. The emulsion, containing micron or submicron sized particles of amniotic membrane, is formulated to increase absorption and overall bioavailability. In one embodiment, the emulsion is sprayed onto an implantable or insertable medical device. In another embodiment, the emulsion is sprayed onto an ocular surface or inserted in or on an ocular device in an individual having or at risk for developing dry eye.

### RELATED APPLICATIONS

This application is a Continuation-In-Part of pending U.S. patent application Ser. No. 10/874,724 filed on Jun. 23, 2004, which is expressly incorporated by reference herein.

### FIELD OF THE INVENTION

The invention is directed to compositions and uses of isolated amniotic membrane with enhanced rigidity for biocompatible devices.

### BACKGROUND

The amniotic membrane is the translucent innermost layer of the three layers forming the fetal membranes, and is derived from the fetal ectoderm. It contributes to homeostasis of the amniotic fluid. At maturity, it is composed of epithelial cells on a basement membrane, which in turn is connected to a thin connective tissue membrane or mesenchymal layer by filamentous strands.

Therapeutic uses of amniotic membrane have included wound coverings and tissues for surgical reconstruction and repair. Ocular uses include ocular repair and coverings for diseased structures (cornea or conjunctiva) and in the management of chemical burns. Grafts of filter paper sheets directionally mounted or adhered with human amniotic membrane containing killed cells have been used. The thin, flexible amniotic membrane is typically sutured to the underlying tissue. To serve as a substrate for corneal or conjunctival epithelial cells, it is positioned with the epithelial (basement membrane) side up and the matrix side down in close apposition to the corneal or episcleral stroma. To protect against inflammation, it is positioned with the epithelial side down and the matrix side towards the palpebral aperture so that the matrix traps inflammatory cells and induces apoptosis. Two amniotic membranes, one with epithelial side up and the other, superimposed on it with the epithelial side down, may be used together.

Other uses of the amniotic membrane are desirable.

### SUMMARY OF THE INVENTION

One embodiment is a biocompatible composition comprising an isolated amniotic membrane treated with at least one consistency-modifying component in an amount sufficient to enhance rigidity of the isolated treated amniotic membrane over non-treated amniotic membrane. The composition may be molded, cured, and shaped to form a free-standing device, such as a shunt, a vessel, a contact lens, etc. Alternatively, the composition may be attached to a device such as an implantable pump or any of the above-mentioned devices. Such a composition may reduce the proliferative response that occurs when devices are implanted or inserted and/or enhance healing.

To enhance rigidity of the amniotic membrane, it may be treated with a polymer and/or a crosslinking agent. The consistency-modifying component may be in an amount ranging from about 0.01%^{w/v} to about 99.99%^{w/v}. In one embodiment, the amniotic membrane is treated with radiation as the consistency-modifying component, resulting in a cross-linked amniotic membrane having enhanced rigidity in the absence of a chemical compound.

The isolated amniotic membrane may be commercially obtained, recombinant, or naturally occurring and sterilized. The concentration of amniotic membrane in the treated composition may range from about 0.1%^{w/v} to about 100%^{w/v}. Polymers may be natural or synthetic and include but are not limited to collagens, mucopolysaccharides, condroitin sulfate, laminin, elastin, fibroin, keratins, hyaluranic acid, integrin, glycosaminoglycans, proteoglycans, fibronectin, hyaluronan, starches, cellulose, agar, alginate, carrageenan, pectin, konjac, gums, chitan, sulfated chitan, chitosan, polylactic acid, polyhydroxyalkanoates, silks, collegin/gelatin, reslin, palamino acids, wheat gluten, casein, soy, zein, serum albumin, cellulose, xanthum, dextran, gellan, levan, curd lan, polygalactosamine, pullulan, elsinan, yeast glucans, acetoglycerides, waxes, emulsan, surfactants, lignin, tannin, humic acid, shellac, polygammaglutamic acid, natural rubber, hydrogel, hilafilcon, hilafilcon B, synthetic polymers made from natural fats and oils, polyethylene, poly(alkylcyanoacrylates), polybutylcyanoacrylates, polyhexylcyanoacrylates, polyethylcyanoacrylate, polyisobutylcyanoacrylate, polycyanoacylate, silica, poly(D,L-lactide-coglycolide, silicone, polyvinylpyrollidone, polyvinylalcohol, polycaprolactone, poly(glycolic acid) (PGA), poly(lactic acid) (PLA), copolymers of PGA and PLA, polydioxananone (PDS), poly(methylmethacrylate) (PMMA), poly(hydroxyethylmethacrylate) (HEMA), glyceroldimethacrylate (GDM), glycerol methacrylate (GMA), copolymerized PMMA with methacryloxypropyl tris(trimethysiloxy silane) (TRIS) PMMA-TRIS, MMA-TRIS doped with fluoromethacrylates, or polydimethylsiloxane (PDMS). The polymer(s) may be crosslinked. The composition is molded, cured, and shaped to form a shunt, a vessel, a lens, etc., or it may be attached to a device without suturing, e.g., coating a shunt, a stent, an insulin pump, etc.

Another embodiment is an insertable or implantable medical device containing in whole or in part an isolated amniotic membrane treated with at least one consistency-modifying component to provide enhanced rigidity over untreated amniotic membrane. The device may contain a drug, such as an agent which either stimulates cell growth or inhibits cell growth, depending upon the desired outcome. This drug, which may be in the form of microcapsules or other controlled-release vehicles, may be included with the consistency-modifying component or with the formed device.

Another embodiment is a method of forming a biocompatible device by molding, curing, and shaping an amniotic membrane treated to have enhanced rigidity to fit an anatomical site requiring the device to form an implantable or insertable device. Treatment may include crosslinking either the amniotic membrane itself and/or one or more added polymers by chemical crosslinking, photocrosslinking, radiation crosslinking, etc. to modify consistency of the amniotic membrane to provide enhanced rigidity. A controlled-release drug may be included in forming the device. An ocular device such as a therapeutic contact lens, a refractive contact lens, an intraocular lens, or a corneal lens inlay may be formed.

Another embodiment is a method to provide a biocompatible implantable or insertable device by enhancing rigidity of an isolated amniotic membrane with a consistency-modifying component under conditions sufficient to enhance rigidity of the amniotic membrane to form a three-dimensional biocompatible implantable or insertable device. The device may be any shape or may be shaped to fit a specific patient and/or a specific anatomical location.

Another embodiment is the use of amniotic membrane formulated as an emulsion or tear drop spray. In this embodiment, amniotic membrane is obtained in any manner as previously described. It is then pulverized and mixed with a physiological solution to form an emulsion. In one embodiment, an amniotic membrane in lipid emulsion is formed. In another embodiment, a lipid emulsion in amniotic membrane is formed. Other solvents that may evaporate may also be added.

In one embodiment, one or more surfactants is added to reduce surface tension around the dispersed drops. The result is a condensed, non-adherent film that reduces coalescence and imparts electrical potential so mutual repulsion occurs. Upon administration to a physiological surface such as an ocular mucous membrane, the emulsion tear drops separate so that the lipid and aqueous components merge with body fluids such as tears.

As will be appreciated by one skilled in the art, one or more medicaments and/or other compounds (e.g., excipients, vitamins, etc.) may also be included. The composition, having or adjusted to have neutral pH and osmolarity, is used. Such a composition may be applied to the eye, mucosa, compromised or intact skin, etc. The composition may also be applied to, or formulated as, a device such as a contact lens, or as a coating for a corneal inlay. It may be used, for example, as a palliative or therapeutic treatment for dry eye, contact lens irritation, etc.

These and other embodiments will be apparent in light of the following figures and detailed description.

### DETAILED DESCRIPTION

Compositions and methods using amniotic membrane with enhanced rigidity for biocompatible devices are disclosed. In one embodiment, the amniotic membrane may be combined with polymers in mixture or admixture. In another embodiment, the amniotic membrane may be treated to crosslink its components to enhance rigidity. Amniotic membranes with enhanced rigidity may be used with biocompatible devices without specific attachment means, such as sutures, and do not require directional orientation. The devices may be made to any shape or size, or to conform to any shape or size, and may be implanted or inserted in the body at one or more anatomical locations. In one embodiment, the devices are for ocular use. The amniotic membrane with enhanced rigidity may comprise the entire device, or may coat, cover, insert in or on, etc., either in whole or in part, a biocompatible device.

The amniotic membrane may be obtained commercially (e.g., Bio-Tissue Inc., Miami Fla.; OKTO Ophtho, Costa Mesa Calif.), with the frozen tissue thawed and rinsed (e.g., in buffered normal saline) before use. It may be obtained postpartum or may be preserved (e.g., in 85% glycerol and stored at 4° C.; in 50% glycerol in tissue culture medium, etc.). Other methods of preservation include lyophilization as described in Burgos et al., J R Soc Med 76:433, 1983 and Steinkogler et al., Klin Monatsbl Augenheilkd 187:359-60, 1985; air drying as described in Martinez Pardo et al., Ann Transplant 4:68-73, 1999, and Rao et al., Arch Surg 116:891-6, 198; glutaraldehyde and polytetrafluoroethylene treatment as described in Muralidharan et al, J Biomed Mater Res 25:1201-9, 1991; cryopreservation as described in Kruse et al., Graefes Arch Clin Exp Opthalmol 238:68-75, 2000, and Kruse et al., Ophthalmology 106:1504-10, 1999; and irradiation as described in Martinez Pardo et al., Ann Transplant 4:68-73, 1999, Rao et al., Arch Surg 116:891-6, 1981, and Tyszkiewicz et al., Ann Transplant 4:85-90, 1999, each of which is expressly incorporated by reference herein. Methods of harvesting, sterilizing, and preserving amniotic membrane are described in Dua et al., Survey of Ophthalmology, 2004, 49: 51-77, which is expressly incorporated herein by reference.

The composition and methods are not limited to the use of amniotic membrane derived from a human source. Amniotic membrane from non-human animals may be used. Recombinant amniotic membrane may also be used, as described in U.S. Patent Application Publication No. 2003/0235580 which is expressly incorporated by reference herein. Such sources permit manufacture of a device independent from harvest of human amniotic membrane, if desired.

Processing and preparation of amniotic membrane occur under sterile conditions. To sterilize the membrane, antibiotics (e.g., a cocktail to cover Gram-negative and Gram-positive bacteria and other microbes), 0.5% silver nitrate, 0.025% sodium hypochlorite, etc. are used in washing and storage solutions. The membrane may be cut into pieces (e.g., about 10 cm×10 cm) and rinsed sequentially for about five minutes in each of 0.5 M dimethyl sulfoxide (DMSO) (4%^{w/v} in 0.01 M phosphate buffered saline PBS), 1.0 M DMSO (8%^{w/v} in 0.01 M PBS), and 1.5 M DMSO (12%^{w/v} in 0.01 M PBS). Alternatively, pieces of the amniotic membrane may be stored in 50% glucerol in Dulbeco's modified Eagle Medium (DMEM, Gibco) or TC-199. The pieces of membrane are usually spread epithelial side up, on nitrocellulose paper before storage in medium. The tissue is stored frozen at −80° C. and released for use only after a normal second serological screening test carried out six months after delivery. Such tissue has been stored and used for up to two years post-delivery.

In one embodiment, this tissue may be processed by trituration or mincing. In another embodiment, this tissue may be pulverized or otherwise rendered into fine particulates. Particles may be micron or submicron size ranges. In one embodiment, particle sizes may range from 1 micron to 100 microns. In another embodiment, particle sizes may range from 10 nm to 100 nm. Amniotic membranes may be sonicated using high frequency sound waves to shear cells, may be subjected to freeze/thaw in a freezer or using dry ice/ethanol where repeated cycles of freezing and thawing disrupt cells through ice crystal formation, may be manually treated using a mortar and pestle, etc.

The resulting powder or particles may be dissolved in one or more biocompatible solvents to create a slurry paste, an emulsion, a physiological solution, etc. One example of a biocompatible solvent is physiological saline. Another example of a biocompatible solvent is commercially available Balanced Salt Solution (BSS®, Alcon Laboratories, Randburg, South Africa) containing per ml 0.64% sodium chloride, 0.075% potassium chloride, 0.048% calcium chloride, 0.03% magnesium chloride, 0.39% sodium acetate, and 0.17% sodium citrate dihydrate, as well as sodium hydroxide and/or hydrochloric acid to adjust pH, and water. Another example of a biocompatible solvent is Ocular Irrigation Solution® (Allergan, Irvine Calif.). Another example of a biocompatible solvent is lactated Ringer's solution. Another example of a biocompatible solvent is a normal saline solution. Another example of a biocompatible solvent is normal saline adjusted to pH 7.4 with sodium bicarbonate.

In one embodiment, amniotic membrane rendered as fine particulates may be formulated as emulsion droplets that can be provided in a spray or tear drop formulation. In this embodiment, amniotic membrane is obtained in any manner as previously described. It is then pulverized and mixed with a physiological solution to form an emulsion. In one embodiment, an amniotic membrane in lipid emulsion is formed. In another embodiment, a lipid emulsion in amniotic membrane is formed. In either embodiment, the lipids may be those used to prepare liposomes, for example dipalmitoyl phosphatidylcholine (DPPC) from egg phosphatidylcholine (PC), a lipid with a low heat of transition. Other lipids that may be used include dimyristoyl phosphatidylcholine (DMPC), distearoylphosphatidylcholine (DSPC), dimyristoyl phosphatidylglycerol (DMPG), and/or sphingomyelin. All are commercially available (e.g., Avanti Polar Lipids, Alabaster Ala.).

In one embodiment, one or more surfactants is added to reduce surface tension around the dispersed drops. Surfactants are surface active groups of amphiphilic molecules manufactured by chemical processes or purified from natural sources. Surfactants, also referred to as surface modifiers, may be excipients and may be anionic, cationic, nonionic, or zwitterionic and are described, for example, in Emulsions: Theory and Practice, Paul Becher, Robert E. Krieger Publishing, Malabar, Fla., 1965; Pharmaceutical Dosage Forms: Dispersed Systems Vol. 1, Martin M. Rigear, Surfactants; and U.S. Pat. No. 5,595,723.

Examples of physiologically compatible surfactants include, but are not limited to, polysorbate-80, octoxynol-40, Carbomer 1342, D-α-tocopherol polyethyleneglycol 1000 succinate (MW=1513) (TPGS), derivatives of vitamin E TPGS including ester and ether linkages of various chemical moieties; polyethylene glycol (MW ranging from 200 to 10,000, e.g., PEG-200, PEG-400, etc.); N-methyl-2-pyrrolidone (NMP); polyvinyl pyrrolidone (PVP); synthetic block copolymers of ethylene oxide and propylene oxide known as poloxamers or Pluronic™ (BASF Performance Chemicals, Parsippany N.J.) which consist of the group of surfactants designated by the CTFA name of Poloxamer 108, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403; a polyethylene glycol 660 hydroxystearate (Solutol HS-15, BASF); ascorbyl-6 palmitate (Roche Vitamins, Nutley N.J.); stearylamine; sucrose fatty acid esters (Mitsubishi Chemicals); custom surfactants such as a vitamin E derivative comprising a peptide bonded polyglutamate attached to the ring hydroxyl and pegylated phytosterol, with or without other peptides; pegylated phospholipids including PEG 2000 or PEG 5000 analogs of phosphatidylethanolamine; phosphocholine surfactants; long chain or high molecular weight (>1000) surfactants including gelatin, casein, lecithin (phosphatides), gum acacia, cholesterol, tragacanth, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, microcrystalline cellulose, magnesium aluminum silicate, triethanolamine, polyvinyl alcohol, and polyvinylpyrrolidene (PVP), tyloxapol (a nonionic liquid polymer of the alkyl aryl polyether alcohol type, also known as superinone or Triton), poloxamers such as Pluronic F68, F77, and F108, polyxamines such as Tetronic 908 (also known as Poloxamine 908, BASF) which is a tetrafunctional block copolymer derived from sequential addition of propylene oxide and ethylene oxide to ethylenediamine, dextran, lecithin, dialkylesters of sodium sulfosuccinic acid such as Aerosol OT which is a dioctyl ester of sodium sulfosuccinic acid (American Cyanamid), Duponol P (a sodium lauryl sulfate, DuPont), Triton X-200 (an alkyl aryl polyether sulfonate, Rohm and Haas), Tween 20 and Tween 80 (polyoxyethylene sorbitan fatty acid esters, ICI Specialty Chemicals), Carbowax 3550 and 934 (polyethylene glycols, Union Carbide); Crodesta F-110 (a mixture of sucrose stearate and sucrose distearate, Croda Inc.), Crodesta SL-40 (Croda, Inc.), and SA90HCO, which is C₁₈H₃₇CH₂(CON(CH₃)CH₂(CHOH)₄ CH₂ OH)₂; low molecular weight (<1000) include stearic acid, benzalkonium chloride, calcium stearate, glycerol monostearate, cetostearyl alcohol, cetomacrogol emulsifying wax, and sorbitan esters; decanoyl-N-methylglucamide; n-decyl.beta-D-glucsopyranoside; n-decyl.beta-D-maltopyranoside; n-dodecyl.beta-D-glucopyranoside; n-dodecyl.beta.-D-maltoside; heptanoyl-N-methylglucamide; n-heptyl-.beta.-D-glucopyranoside; n-heptyl.beta.-D-thioglucoside; n-hexyl.beta.-D-glucopyranoside; nonanoyl-N-methylglucamide; n-noyl.beta.-D-glucopyranoside; octanoyl-N-methylglucamide; n-octyl-.beta.-D-glucopyranoside; octyl.beta.-D-thioglucopyranoside; isononylphenoxypoly (glycidol) (also known as Olin-10G or Surfactant 10-G, Olin Chemicals, Stamford Conn.), and the like. Such surfactants are known pharmaceutical excipients and their compositions, formulations, uses, etc. are described in detail in the Handbook of Pharmaceutical Excipients, published jointly by the American Pharmaceutical Association and The Pharmaceutical Society of Great Britain, the Pharmaceutical Press, 1986.

Examples of lipids that may be used in formulating the emulsion include, but are not limited to, mineral oils (e.g., Drakeol-15, Drakeol-35), castor oil, vitamins (e.g., vitamin A, vitamin E). Lipids may be those used to prepare liposomes, for example dipalmitoyl phosphatidylcholine (DPPC) from egg phosphatidylcholine (PC), a lipid with a low heat of transition. Other lipids that may be used include dimyristoyl phosphatidylcholine (DMPC), distearoylphosphatidylcholine (DSPC), dimyristoyl phosphatidylglycerol (DMPG), and/or sphingomyelin. All are commercially available (e.g., Avanti Polar Lipids, Alabaster Ala.).

The result is a condensed, non-adherent film that reduces coalescence and imparts electrical potential so mutual repulsion occurs. Upon administration to a physiological surface such as an ocular mucous membrane, the emulsion tear drops separate so that the lipid and aqueous components merge with body fluids such as tears. Without being bound by a specific theory, the electrostatic attraction occurring between positively charged emulsion droplets and negatively charged biological membranes results in adsorption of the droplets onto the cell surface. This results in increased absorption and overall bioavailability of the composition. The compositions thus formulated may be used in injectable, oral, topical, ocular, etc. routes of administration.

As will be appreciated by one skilled in the art, one or more medicaments and/or other compounds (e.g., excipients, vitamins such as zeaxanthin, lutein, etc.) may also be included. Medicaments include, but are not limited to, anti-inflammatory agents such as macrolides, steroids, non-steroidal anti-inflammatory drugs (NSAIDS), antibiotics, anti-prostaglandins, anti-angiogenic agents (anti-vascular endothelial growth factor (anti-VEGF) agents), etc. Anti-angiogenic agents include, but are not limited to, TNP470, integrin av antagonists, 2-methoxyestradiol, paclitaxel, P38 mitogen activated protein kinase inhibitors, anti-VEGF siRNA, and sunitinib maleate (Sutent®/SU11248). Examples of anti-inflammatory agents include, but are not limited to, colchicine; a steroid such as triamcinolone (Aristocort®; Kenalog®), anecortave acetate (Alcon), betamethasone (Celestone®), budesonide cortisone, dexamethasone (Decadron-LA®; Decadron® phosphate; Maxidex® and Tobradex® (Alcon)), hydrocortisone methylprednisolone (Depo-Medrol®, Solu-Medrol®), prednisolone (prednisolone acetate, e.g., Pred Forte® (Allergan), Econopred and Econopred Plus® (Alcon), AK-Tate® (Akorn), Pred Mild® (Allergan), prednisone sodium phosphate (Inflamase Mild and Inflamase Forte® (Ciba), Metreton® (Schering), AK-Pred® (Akorn)), fluorometholone (fluorometholone acetate (Flarexe (Alcon), Eflone®), fluorometholone alcohol (FML® and FML-Mild®, (Allergan), Fluor OP®), rimexolone (Vexole (Alcon)), medrysone alcohol (HMS® (Allergan)), lotoprednol etabonate (Lotemaxe and Alrexe (Bausch & Lomb), and 11-desoxcortisol; an anti-prostaglandin such as indomethacin; ketorolac tromethamine; ((±)-5-benzoyl-2,3-dihydro-1H-pyrrolizine-1-carboxylic acid, a compound with 2-amino-2-(hydroxymethyl)-1,3-propanediol (1:1) (Acular® Allegan), Ocufen® (flurbiprofen sodium 0.03%), meclofenamate, fluorbiprofen, and the pyrrolo-pyrrole group of non-steroidal anti-inflammatory drugs. Examples of macrolides include, but are not limited, to sirolimus (rapamycin), pimocrolous, tacrolimus (FK506), cyclosporine (Arrestase), everolimus 40-O-(2-hydroxymethylenrapamycin), ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, biolimus, ABT-578 (methylrapamycin), and derivatives of rapamycin such as temsirolimus (CCI-779, Wyeth) and AP23573 (Ariad). Examples of NSAIDS include, but are not limited to, non-steroidal anti-inflammatory drug such as derivatives of acetic acid (e.g. diclofenac and ketorolac (Toradol®, Voltaren®, Voltaren-XR®, Cataflam®)), salicylate (e.g., aspirin, Ecotrin®), proprionic acid (e.g., ibuprofen (Advil®, Motrin®, Medipren®, Nuprin®)), acetaminophen (Tylenol®), aniline (e.g., aminophenolacetaminophen, pyrazole (e.g., phenylbutazone), N-arylanthranilic acid (fenamates) (e.g., meclofenamate), indole (e.g., indomethacin (Indocin®, Indocin-SR®)), oxicam (e.g., piroxicam (Feldene®)), pyrrol-pyrrole group (e.g., Acular®), antiplatelet medications, choline magnesium salicylate (Trilisate®), cox-2 inhibitors (meloxicam (Mobic®)), diflunisal (Dolobid®), etodolac (Lodine®), fenoprofen (Nalfon®), flurbiprofen (Ansaid®), ketoprofen (Orudis®, Oruvail®), meclofenamate (Meclomen®), nabumetone (Relafen®), naproxen (Naprosyn®, Naprelan®, Anaprox®, Aleve®), oxaprozin (Daypro®), phenylbutazone (Butazolidine®), salsalate (Disalcid®, Salflex®), tolmetin (Tolectin®), valdecoxib (Bextra®), sulindac (Clinoril®), and flurbiprofin sodium (Ocufen®), an MMP inhibitor such as doxycycline, TIMP-1, TIMP-2, TIMP-3, TIMP-4; MMP1, MMP2, MMP3, Batimastat (BB-94), TAPI-2,10-phenanthroline, and marimastat. Examples of anti-platelet derived growth factor (PDGF) compounds include, but are not limited to, imatinib mesylate (Gleevec®), sunitinib malate (Sutent®) which has anti-PDGF activity in addition to anti-VEGF activity, and/or anti-leukotriene(s) such as genleuton, montelukast, cinalukast, zafirlukast, pranlukast, zileuton, BAYX1005, LY171883, and MK-571 to account for the involvement of factors besides VEGF in neovascularization. The composition may additionally contain other agents including, but not limited to, transforming growth factor β (TGFβ), interleukin-10 (IL-10), aspirin, a vitamin, and/or an anti-proliferative agent, an antimicrobial, a growth stimulatory factor, a growth inhibitory factor, a hormone, an antibody, or an immunomodulator.

In one embodiment, basement membrane components of the amniotic membrane may be separated to create derivitized amniotic membrane, which may be treated by one or more of the methods as described.

The composition, having or adjusted to have neutral pH and osmolarity, is used. In one embodiment, such a composition is applied to the eye. In another embodiment, such a composition is applied to a wound, including abrasions and contusions. In another embodiment, such a composition is applied to a mucosa (e.g., nasal, vaginal, oral, etc.). In another embodiment, such a composition is applied skin, including both intact skin and compromised skin. The composition may also be applied to, or formulated with or as a device such as an ocular device. Examples of such an ocular device include, but are not limited to, a contact lens, or a coating for a corneal inlay. Such ocular devices may be used, for example, as a palliative or therapeutic treatment for dry eye, contact lens irritation, etc.

The aminotic membrane in a fine particulate formulation retains its palliative and/or therapeutic effect, e.g., reducing inflammation, maintaining osmotic pressure, etc., without evaporation.

The dissolved or suspended amniotic membrane compositions, in the form of a slurry or in another form, is molded, cured, and treated to enhance its rigidity. In one embodiment, one or more crosslinking agents are added to enhance rigidity. In another embodiment, one or more biocompatible polymers are added and may be crosslinked and/or cured to enhance rigidity. In another embodiment, no additional substance is added but the amniotic membrane is treated such that its components are crosslinked to enhance rigidity. This may be done, for example, by treating with radiation (e.g., photocrosslinking), where the radiation serves as the consistency-modifying component to enhance rigidity.

The resulting amniotic membrane with modified consistency has less propensity to tear upon manipulation and may be sufficiently rigid to serve as a device itself, or to be provided to a pre-formed device. In various embodiments, the concentration of amniotic membrane in the composition may range from about 0.01%^{w/v} of the composition to about 99.99%^{w/v} of the composition, about 0.1%^{w/v} of the composition to about 99.9%^{w/v} of the composition, from about 1.0%^{w/v} of the composition to about 99.0%^{w/v} of the composition, or from about 10.0%^{w/v} of the composition to about 90.0%^{w/v} of the composition. As one example, the composition may contain about 50%^{w/v} amniotic membrane and about 50%^{w/v} of one or more polymers. As another example, the composition may contain about 60%^{w/v} amniotic membrane and about 40%^{w/v} of one or more polymers. As another example, the composition may contain about 50%^{w/v} amniotic membrane and about 50%^{w/v} crosslinking agent(s). Any combination of amniotic membrane and rigidity-enhancing agent(s) may be used that increases the rigidity of amniotic membrane over its unmodified state. This may be evaluated, for example, by assessing deflection (i.e., flexibility or bending) as a load is applied to the amniotic membrane, by optical or other means as known to one skilled in the art. The thus-modified amniotic membrane has a consistency more readily manipulated than that of non-modified amniotic membrane, which has a consistency resembling wet tissue paper.

In one embodiment, polymers may be used. Polymers include, but are not limited to, those that form structural components of the cell, including polysaccharides and polypeptides. Examples are the families of collagen (e.g., collagen types I, III, IV, V, VII), mucopolysaccharides, condroitin sulfate, fibronectin, laminins (e.g., laminins-1, -5, -6, -7) and other attachment polymers, elastin, fibroin, keratins, hyaluranic acid, integrin, glucosaminoglycan, proteoglycans (e.g., biglycan, decorin), fibronectin, hyaluronan, etc. Biopolymers may be used, such as those derived from crops, shellfish, algae, etc., including plant/algal polysaccharides such as starches, cellulose, agar, alginate, carrageenan, pectin, konjac, guar and other gums; animal polysaccharides such as chitan, sulfated chitan, chitosan; polyesters such as polylactic acid, polyhydroxyalkanoates; proteins such as silks, collegin/gelatin, elastin, reslin, palamino acids, wheat gluten, casein, soy, zein, serum albumin; bacterial polysaccharides such as cellulose, xanthum, dextran, gellan, levan, curd Ian, polygalactosamine; fungal polysaccharides such as pullulan, elsinan, yeast glucans; lipids such as acetoglycerides, waxes, emulsan, surfactants; polyphenols such as lignin, tannin, humic acid; shellac, polygammaglutamic acid, natural rubber, etc. Synthetic polymers may be used and include, but are not limited to, hydrogel, hilafilcon, hilafilcon B, synthetic polymers made from natural fats and oils (e.g., nylob from castor oil), polyethylene, poly(alkylcyanoacrylates), polybutylcyanoacrylates, polyhexylcyanoacrylates, polyethylcyanoacrylate, polyisobutylcyanoacrylate, polycyanoacylate, silica, poly(D,L-lactide-coglycolide, silicone, polyvinylpyrollidone, polyvinylalcohol, poly(glycolic acid) (PGA), poly(lactic acid) (PLA), copolymers of PGA and PLA, polycaprolactone, polydioxananone (PDS), poly(methylmethacrylate) (PMMA), poly(hydroxyethylmethacrylate) (HEMA), glyceroldimethacrylate (G DM), glycerol methacrylate (G MA), copolymerized PMMA with methacryloxypropyl tris(trimethylsiloxy silane) (TRIS) (PMMA-TRIS), MMA-TRIS doped with fluoromethacrylates; polydimethylsiloxane (PDMS), etc. Properties, vendors, and functions of such polymers are known to one skilled in the art.

One or more of the same or different polymers may be included in the mixture. The specific formulation may depend upon device specific factors such as its size, function, site of implantation or insertion, etc., patient-specific factors such as presence of an inflammatory response, underlying pathology, age, etc., as well as other factors such as ease of formulation, etc. For example, hydrogels are polyelectrolytes and are water soluble. To render hydrogels insoluble they are crosslinked, with the degree of crosslinking, quantified in terms of crosslink density, affecting their swelling and other characteristics. The polymers may be obtained as commercial products (e.g., Sigma Aldrich, St. Louis Mo.), and may be naturally occurring or synthetic as known to one skilled in the art.

The resultant amniotic membrane/polymer mixture may be molded, crosslinked, and/or cured to any shape, size, dimension, structure, etc. as needed. Curing may occur upon application of light with a photo-initiator, by using chemical crosslinking, and/or the mixture may be self-curing, for example, by including a redox initiatior. In one embodiment, it may be formulated as a covering, either total or partial, on devices such as a refractive contact lens, or a therapeutic contact lens, or an intraocular lens. In another embodiment, it may be formulated as an inlay for implanting under the corneal epithelium or in the stroma to achieve a desired refractive surface of the cornea. It may contain factors promoting epithelial cell growth that include, but are not limited to, nerve growth factor. Additionally or alternatively, it may contain hormones or factors that help to reduce neovascularization, such as pigment epithelial-derived growth factor (PEGF) that inhibits VEGF-F induced neovascularization. The device may be shaped to produce a negative surface for the cornea after implantation, or a positive surface, a toric surface, or a multifocal surface, as known to one skilled in the art. In another embodiment, it may be casted to an appropriate shape, such as a globe, tube, rod, thin plate, etc.

In one embodiment, either the amniotic membrane composition without a polymer, or an amniotic membrane and polymer composition may be crosslinked. Crosslinking enhances stability and durability, and may be used to achieve a desired shape. Crosslinking is the formation of chemical links between molecular chains to form a three-dimensional network of connected molecules, and can increase the density of the composition to improve its strength and hardness, that is, to enhance its rigidity. Methods, reagents, and parameters are selected to suit the desired application, as known to one skilled in the art. For example, known commercially available chemical crosslinking agents (e.g., Sigma Aldrich, St. Louis Mo.; Pierce, Rockford Ill.) such as glutaraldehyde, lysine oxidase, group specific crosslinkers such as the amine-sulfhydryl crosslinker succinimidyl-6-[βmaleimidopropion-amido]hexanoate (SMPH) or the hydroxyl and sulfhydryl reactive crosslinker N-[p-maleimidophenyl]isocyanate (PMPI), or the photoreactive crosslinker N-sulfosuccinimidyl(4-azidophenyl)-1,3′-dithiopropionate (sulfo-SADP), etc. may be added for chemical crosslinking, and/or the composition may be irradiated with ultraviolet light for photocrosslinking. Polyethylene, depending upon its processing, may be elastic and flexible, or hard and smooth. Low density polyethylene may be formulated as a tube, such as a synthetic blood vessel. In contrast, high and ultra high density polyethylene may be used where a non-flexible device is required. Crosslinking monomers such as derivatives of ethylene glycol di(meth)acrylate; methylenebisacrylamide; divinylbenzene; (hydroxydimethoxyethyl)acrylamide may be used in some embodiments. Any of the above-described devices are free-standing and thus are independent from further attachment, such as suturing that must be performed to cover both the cornea and the conjunctiva. Because the modified amniotic membrane has enhanced rigidity, that is, it is sturdier and less flimsy than unmodified amniotic membrane, it can be readily handled and implanted.

Various embodiments of the invention may be used. In one embodiment, the inventive device may be implanted or inserted under the retina to promote cellular growth over the retina in conditions when retinal pigment epithelial cells are lost, such as in age-related macular degeneration. In another embodiment, the inventive device may be implanted or inserted to provide corneal endothelial cells to replace or repair a damaged cornea. In another embodiment, tissue culture techniques, known to those skilled in the art, are used to generate cell growth on the inventive device prior to transplant. The inventive device provides a stable platform where cells can adhere and properly be implanted because the membrane does not readily fold over itself with simple manipulation, as may occur with amniotic membrane alone.

In one embodiment, the treated amniotic membrane may be provided on an exterior surface of an implantable or insertable device. In another embodiment, the treated amniotic membrane may be provided on the luminal (internal) surface of synthetic vessels. One example is synthetic arteries or veins made from Gortex or any other material. The membrane may act as a scaffold to promote endothelial cell growth, and can act as a replacement vessel in repairing occluded or damaged vessels. Uses include, but are not limited to, blood vessels that are damaged after surgical manipulation (e.g., stent implantation), and synthetic vessels to heal or repair a damaged urethra or ureter, in limb replacement surgery, etc.

In another embodiment, the inventive device may carry drugs. For example, a device such as a contact lens of an amniotic membrane polymer composition may contain one or more agents depending upon the desired outcome. The contact lens may include antibodies, antimicrobials antiproliferative agents, chemotherapeutic agents, cell mediators, immunomodulators, growth stimulatory factors, growth inhibitory factors, hormones, etc. Another type of device, either with or without drugs, is implantable under the conjunctiva, inside the eye, under the skin, under the eye lid, etc. The drug(s) may be in or on microcapsules, microspheres, liposomes, nanoparticles, etc. for a slow release delivery system by methods known to one skilled in the art and as described in U.S. Pat. No. 5,185,152 and published U.S. patent application Ser. Nos. 10/289,772 and 10/454,836, each of which is expressly incorporated by reference herein.

Any implantable or insertable device may be coated externally with the amniotic membrane, and/or with the amniotic membrane/polymer composition. Such an embodiment may take advantage of the amniotic membrane's ability to reduce a tissue proliferative response, which desirably may eliminate vascularization and rejection of various grafts. Such an embodiment may also reduce the undesirable excessive tissue response to the device itself. In one embodiment, the amniotic membrane, alone or combined with polymers as described, is used as an at least partial covering or component of glaucoma shunts. Patients with glaucoma may have a glaucoma shunt implanted to connect the intraocular cavity to the subconjunctival space to drain excess amounts of intraocular fluid, and hence reduce intraocular pressure. Glaucoma shunts often become heavily encapsulated in a fibrous material, severely reducing or even restricting fluid drainage. A similar encapsulation problem also occurs with drug delivery devices implanted in the body, such as an insulin or morphine pump. Incorporating modified amniotic membrane with the device enhances proper flow of the drug from the device to the tissue and circulation.

Other variations or embodiments of the invention will also be apparent to one of ordinary skill in the art from the above descriptions. Thus, the forgoing embodiments are not to be construed as limiting the scope of this invention.

What is claimed is:

1. A physiological composition comprising amniotic membrane at a concentration ranging from: about 0.1%^{w/v} to about 100%^{w/v} in at most micron sized particulates and at least one lipid resulting in an emulsion.

2. The composition wherein the amniotic membrane particle sizes range from about 1 nm to 100 microns.

3. The composition of claim 1 wherein the excipient is selected from the group consisting of saline, a buffer, a salt, an osmolarity adjusting agent, a polymer, a crosslinking agent, and combinations thereof.

4. The composition of claim 1 further comprising a medicament.

5. The composition of claim 1 wherein the emulsion is provided on an ocular shunt, a corneal inlay, or an ocular lens.

6. A physiologic method comprising providing to an individual a composition comprising at least one lipid and amniotic membrane ranging from about 0.1%^{w/v} to about 100%^{w/v} of the composition, where the amniotic membrane is in at most micron sized particulates resulting in an emulsion for contact with a physiologic surface.

7. The method of claim 6 wherein the composition is injected, provided orally, provided topically, or provided ocularly.

8. The method of claim 6 wherein the composition is provided topically to a mucosal surface of the eye.

9. The method of claim 6 wherein the amniotic membrane composition is provided for insertion in an eye.

10. The method of claim 6 wherein the composition further comprises a medicament.

11. The method of claim 6 wherein the composition is provided to an individual having or at risk for developing dry eye.

12. A method to reduce a proliferative response to an implanted or inserted medical device comprising spraying at least a portion of the medical device with an amniotic membrane emulsion to provide a physiological surface and thereby reduce a proliferative response to the implanted or inserted medical device.
